(19) United States
(12) Patent Application Publication
(10) Pub. No.: US 2014/0309520 A1
(43) Pub. Date: Oct. 16, 2014

(54) MAGNETIC RESONANCE APPARATUS AND METHOD FOR CALCULATING PERFUSION FRACTION

(71) Applicant: GE MEDICAL SYSTEMS GLOBAL TECHNOLOGY COMPANY, LLC, Waukesha, WI (US)

(72) Inventor: Sangwoo Lee, Seoul (KR)

(73) Assignee: GE MEDICAL SYSTEMS GLOBAL TECHNOLOGY COMPANY, LLC, Waukesha, WI (US)

(21) Appl. No.: 14/248,786

(22) Filed: Apr. 9, 2014

(30) Foreign Application Priority Data
Apr. 11, 2013 (JP) 2013-083275

(51) Int. Cl. G01R 33/563; A61B 5/026; G01R 33/56; G01R 33/50

(52) U.S. Cl. CPC G01R 33/56366; G01R 33/50; A61B 5/0263; G01R 33/5602
USPC 600/419; 600/410

(57) ABSTRACT

A magnetic resonance apparatus is provided. The magnetic resonance apparatus includes a scanning unit configured to execute a plurality of T2-weighted sequences having different echo times, and a plurality of diffusion-weighted sequences having different b factors, a first calculating unit configured to calculate a T2 value based on a plurality of T2-weighted images obtained by the plurality of T2-weighted sequences, a second calculating unit configured to calculate a perfusion fraction where the T2 value is not taken into account based on a first T2-weighted image in the plurality of T2-weighted images and a plurality of diffusion-weighted images obtained by the plurality of diffusion-weighted sequences, and a third calculating unit configured to calculate a perfusion fraction where the T2 value has been taken into account based on the T2 value calculated by the first calculating unit, and based on the perfusion fraction calculated by the second calculating unit.

### CROSS REFERENCE TO RELATED APPLICATIONS

This application claims the benefit of Japanese Patent Application No. 2013-083475 filed Apr. 11, 2013, which is hereby incorporated by reference in its entirety.

### BACKGROUND OF THE INVENTION

The present invention relates to a magnetic resonance apparatus that executes diffusion-weighted sequences and a program to be applied to the magnetic resonance apparatus.

A diffusion-weighted sequence that uses an IVIM (IntraVoxel Incoherent Motion) model is known (see for example, Japanese Patent Application Laid-Open No. Hei 10-248824).

As a model indicative of a signal value of a diffusion-weighted image, the following IVIM model using a b factor is known.

S/S₀=f·exp {−b·(D+D*)}+(1−f)·exp(−b·D) Formula (1)

where
S is a Signal value with an arbitrary b factor,
S₀ is a Signal value when b=0,
D is an Apparent diffusion coefficient by a true diffusion component,
D* is an Apparent diffusion coefficient by a pseudo diffusion component, and f is a Perfusion fraction.

Recently, it was indicated that the perfusion fraction (Perfusion Fraction) f depends on an echo time TE (See, Lemke et al., An in vivo verification of the intravoxel incoherent motion effect in diffusion-weighted imaging of the abdomen. Magn. Res. Med. 2010; 64: 1580-1585). The reason why the perfusion fraction f depends on the echo time TE is related to a difference in T1 between a parenchyma and blood and a difference in T2 between the parenchyma and the blood. There is a report that in a 5 T (Tesla) magnetic resonance apparatus, the T2 value of the liver is 15.8% of the T2 value of the blood. Examples of the reported T1 and T2 of the liver and T1 and T2 of the blood are shown in the following table.

TABLE 1

| | Liver | Blood |
|---|---|---|
| T1 | 586 ms | 1446 ms |
| T2 | 46 ms | 290 ms |

In a case where the echo time TE has a value close to the T2 value or the T1 value of the parenchyma, it sometimes occurs that an estimated value of the perfusion fraction f becomes too large owing to a difference in T2 value (or T1 value) between the parenchyma and the blood.

Therefore, it is desired to reduce an error of estimation of the perfusion fraction.

### BRIEF DESCRIPTION OF THE INVENTION

In a first aspect, a magnetic resonance apparatus is provided. The magnetic resonance apparatus includes a scanning unit for executing a plurality of T2-weighted sequences having different echo times and a plurality of diffusion-weighted sequences having different b factors, a first calculating unit for calculating a T2 value on the basis of a plurality of T2-weighted images obtained by the aforementioned plurality of T2-weighted sequences, a second calculating unit for calculating a perfusion fraction that the T2 value is not taken into account on the basis of a first T2-weighted image in the aforementioned plurality of T2-weighted images and a plurality of diffusion-weighted images obtained by the aforementioned plurality of diffusion-weighted sequences, and a third calculating unit for calculating a perfusion fraction that the T2 value has been taken into account on the basis of the T2 value calculated by the aforementioned first calculating unit, and the perfusion fraction calculated by the aforementioned second calculating unit.

In a second aspect, a program is provided. The program is configured to make a computer execute a first calculation process of calculating a T2 value on the basis of a plurality of T2-weighted images obtained by a plurality of T2-weighted sequences having different echo times, a second calculation process of calculating a perfusion fraction that the T2 value is not taken into account on the basis of a first T2-weighted image in the aforementioned plurality of T2-weighted images, and a plurality of diffusion-weighted images obtained by a plurality of diffusion-weighted sequences having different b factors, and a third calculation process of calculating a perfusion fraction that the T2 value has been taken into account on the basis of the T2 value calculated by the aforementioned first calculation process, and the perfusion fraction calculated by the aforementioned second calculation process.

Since the perfusion fraction that the T2 value has been taken into account can be calculated, the error of estimation of the perfusion fraction can be reduced.

Further advantages of the systems and methods described herein will be apparent from the following description of exemplary embodiments as illustrated in the accompanying drawings.

### BRIEF DESCRIPTION OF THE DRAWINGS

FIG. 1 is a schematic diagram of a magnetic resonance apparatus of an exemplary embodiment.

FIG. 2 is a diagram schematically showing a part to be imaged.

FIG. 3 is a diagram showing sequences to be executed in the exemplary embodiment.

FIG. 4 is a diagram showing an operation flow of the MR apparatus of the exemplary embodiment.

FIG. 5 is a diagram schematically showing fetched signal values.

FIG. 6 is a diagram schematically showing a state after a model of a signal value expressed by a formula (7) has been subjected to fitting.

FIG. 7 is a diagram schematically showing fetched signal values.

FIG. 8 is a diagram schematically showing D, D* and A which have been calculated by fitting.

FIG. 9 is an explanatory diagram of a method of calculating a perfusion fraction f′.

FIGS. 10A and 10B are diagrams showing results of simulation.

### DETAILED DESCRIPTION OF THE INVENTION

Although exemplary embodiments will be described in the following, the disclosure is not limited to the following exemplary embodiments.

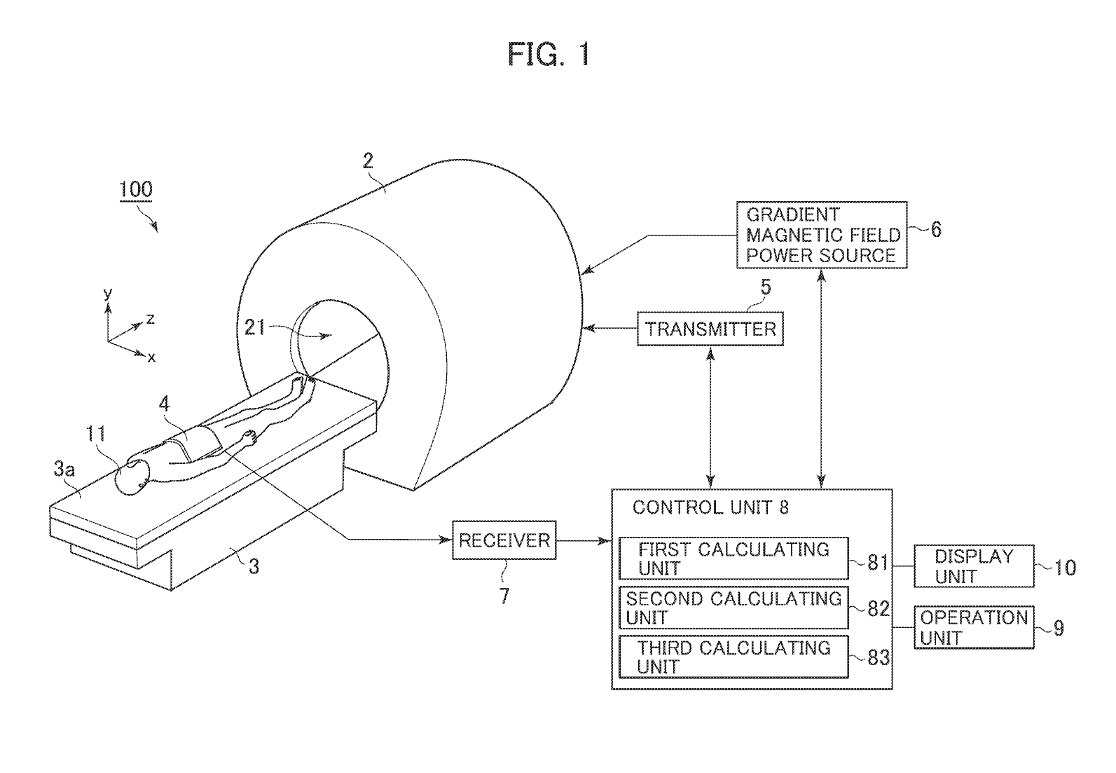

FIG. 1 is a schematic diagram of a magnetic resonance apparatus in an exemplary embodiment.

A magnetic resonance apparatus (hereinafter, referred to as an “MR apparatus” where MR is Magnetic Resonance) 100 has a magnet 2, a table 3, a receiving coil 4 and so forth.

The magnet 2 has a bore 21 in which a subject 11 is contained. In addition, a superconductive coil, a gradient coil, an RF coil and so forth are built in the magnet 2.

The table 3 has a cradle 3a for supporting the subject 11. The cradle 3a is configured to be movable within the bore 21. The subject 11 is conveyed into the bore 21 by the cradle 3a.

The receiving coil 4 is attached to the subject 11. The receiving coil 4 receives a magnetic resonance signal from the subject 11.

The MR apparatus 100 further has a transmitter 5, a gradient magnetic field power source 6, a receiver 7, a control unit 8, an operation unit 9, a display unit 10 and so forth.

The transmitter 5 supplies a current to the RF coil, and the gradient magnetic field power source 6 supplies a current to the gradient coil.

The receiver 7 executes signal processing such as wave detection and so forth on signals which have been received from the receiving coil 4.

The control unit 8 controls operations of respective parts of the MR apparatus 100 so as to implement various operations of transferring required information to the display unit 10, reconstructing an image on the basis of data received from the receiver 7 and so forth of the MR apparatus 100. The control unit 8 has a first calculating unit 81 to a third calculating unit 83 and so forth.

The first calculating unit 31 calculates a T2 value.

A second calculating unit 82 calculates a perfusion fraction (designated by a symbol A in the later described formula (4)) where the T2 value is not taken into account.

The third calculating unit 83 calculates a perfusion fraction (designated by a symbol f′ in the later described formula (5)) where the T2 value has been taken into account.

The control unit 8 is one example of the first calculating unit 81 to the third calculating unit 83 and functions as these units by executing a predetermined program.

The operation unit 9 is operated by an operator to input various pieces of information into the control unit 8. The display unit 10 displays various pieces of information.

The MR apparatus 100 is configured as mentioned above.

In the exemplary embodiment, the perfusion fraction is calculated. In the following, a way of calculating the perfusion fraction in the exemplary embodiment will be described.

As a method of calculating the perfusion fraction, use of a signal model indicated in the formula (1) is conceivable. However, in the signal model in the formula (1), it sometimes occurs that the estimated value of the perfusion fraction f becomes too large due to the difference in T2 value (or T1 value) between the parenchyma and the blood. Thus, as the method of calculating the perfusion fraction, use of an IVIM model that a T1 relaxation effect and a T2 relaxation effect have been taken into account is conceivable. The IVIM model that the T1 relaxation effect and the T2 relaxation effect have been taken into account is proposed by Lemke et al. (Reference: Lemke et al., An in vivo verification of the intravoxel incoherent motion effect in diffusion-weighted imaging of the abdomen. Magn. Res. Med. 2010; 64: 1580-1585). In this model, the signal value of a diffusion-weighted image is expressed by the following formula.

$$Formula ( 2 ) \\ \frac{S}{{S}_{0}} = \frac{( 1 - {f}^{′} ) ( 1 - exp ( - \frac{TR}{T {1}_{tiss}} ) ) exp ( - \frac{TE}{T {2}_{tiss}} - bD ) + {f}^{′} ( ( 1 - exp ( - \frac{TR}{T {1}_{b 1}} ) ) exp ( - \frac{TE}{T {2}_{b 1}} - b ( D + {D}^{*} ) ) )}{( 1 - {f}^{′} ) exp ( - \frac{TE}{T {2}_{tiss}} ) ( 1 - exp ( - \frac{TR}{T {1}_{tiss}} ) ) + {f}^{′} exp ( - \frac{TE}{T {2}_{b 1}} ) ( 1 - exp ( - \frac{TR}{T {1}_{b 1}} ) )} \qquad$$

where

f′ is a Perfusion fraction where the T1 value and T2 value have been taken into account,

TR is a Repetition time,

TE is an Echo time,

T1ₜᵢₛₛ is a T1 value of a parenchyma,

T1_{bl} is a T1 value of blood,

T2ₜᵢₛₛ is a T2 value of the parenchyma, and

T2_{bl} is a T2 value of the blood.

Since the signal model of the formula (2) includes the T1 value and the T2 value, the perfusion fraction f′ where the T1 value and the T2 value have been taken into account can be calculated. Therefore, the perfusion fraction f′ which is small in error of estimation can be obtained. However, since in the signal model of the formula (2), it is necessary to obtain both of the T1 value and the T2 value in order to calculate the perfusion fraction f′, it is not practical. Thus, in the exemplary embodiment, the formula (2) is simplified so as to readily calculate the perfusion fraction f′. In the following, a way of simplifying the formula (2) will be described.

In a case where a sequence for acquiring the diffusion-weighted image by general EPI is used, TR is comparatively long and, for example, TR=6.5 s. In this case, since T1s of the blood and the parenchyma are considerably shorter than TR, the formula (2) can be expressed as follows.

$$\frac{S}{{S}_{0}} = \frac{( 1 - {f}^{′} ) exp ( - \frac{TE}{T {2}_{tiss}} - bD ) + {f}^{′} exp ( \begin{matrix}- \frac{TE}{T {2}_{b 1}} - \\ b ( D + {D}^{*} )\end{matrix} )}{( 1 - {f}^{′} ) exp ( - \frac{TE}{T {2}_{tiss}} ) + {f}^{′} exp ( - \frac{TE}{T {2}_{b 1}} )} \qquad Formula ( 3 )$$

In addition, the formula (3) can be expressed as follows.

$$\begin{matrix}\frac{S}{{S}_{0}} = ( 1 - A ) exp ( - bD ) + A exp ( - b ( D + {D}^{*} ) ) \\ Where & Formula ( 4 ) \\ A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )} & Formula ( 5 )\end{matrix}$$

Comparing the formula (1) with the formula (4), it can be seen that the perfusion fraction f in the formula (1) is indicated by a coefficient A in the formula (4). The coefficient A in the formula (4) is expressed by the formula (5). Although T2_{bl} and T2ₜᵢₛₛ are included in the formula (5), T1_{bl} and T1ₜᵢₛₛ are deleted, and therefore it can be seen that the perfusion fraction f′ can be calculated by using the formula (5) even if T1_{bl} and T1ₜᵢₛₛ are not known.

Incidentally, although T2_{bl} and T2ₜᵢₛₛ are included in the formula (5), T1_{bl} and T1ₜᵢₛₛ are deleted, and therefore f′ in the formula (5) indicates the perfusion fraction that the T2 value has been taken into account. On the other hand, since the coefficient A in the formula (5) is the same as the perfusion fraction f of the model of the formula (1), the coefficient A itself indicates the perfusion fraction where the T2 value is not taken into account.

In the exemplary embodiment, various parameters such as the perfusion fraction f′ and so forth are calculated by using the formula (4) and the formula (5). In the following, a scan to be executed when calculating these parameters will be described.

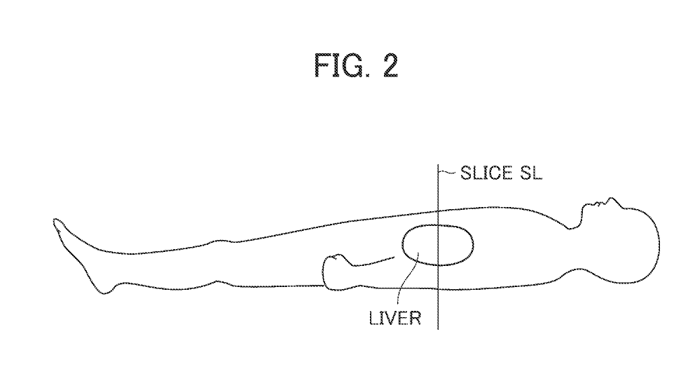

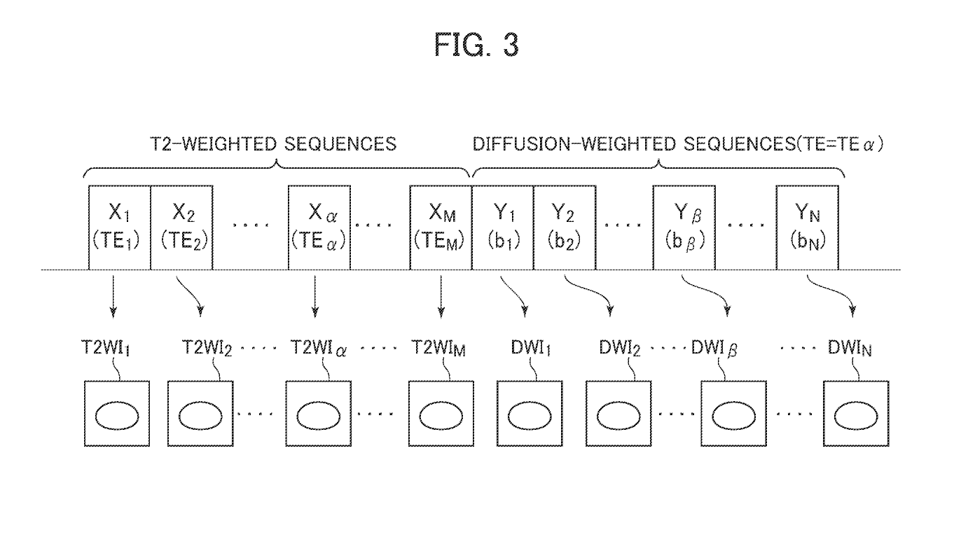

FIG. 2 is a diagram schematically showing a part to be imaged. FIG. 3 is a diagram schematically showing the scan to be executed in the exemplary embodiment.

In the exemplary embodiment, the scan including M T2-weighted sequences X₁ to X_{M}, and N diffusion-weighted sequences Y₁ to Y_{M} is executed. Incidentally, although in general a plurality of slices is set for the part to be imaged, it is supposed that only one slice SL is set for the part to be imaged for the convenience of description in the exemplary embodiment.

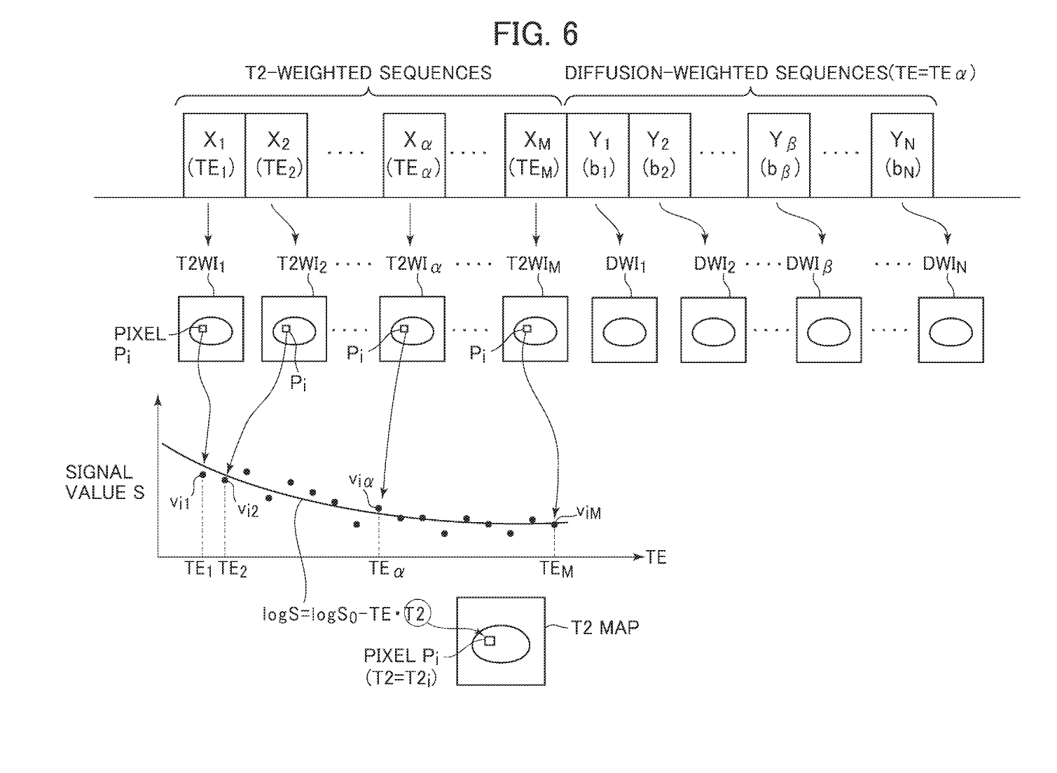

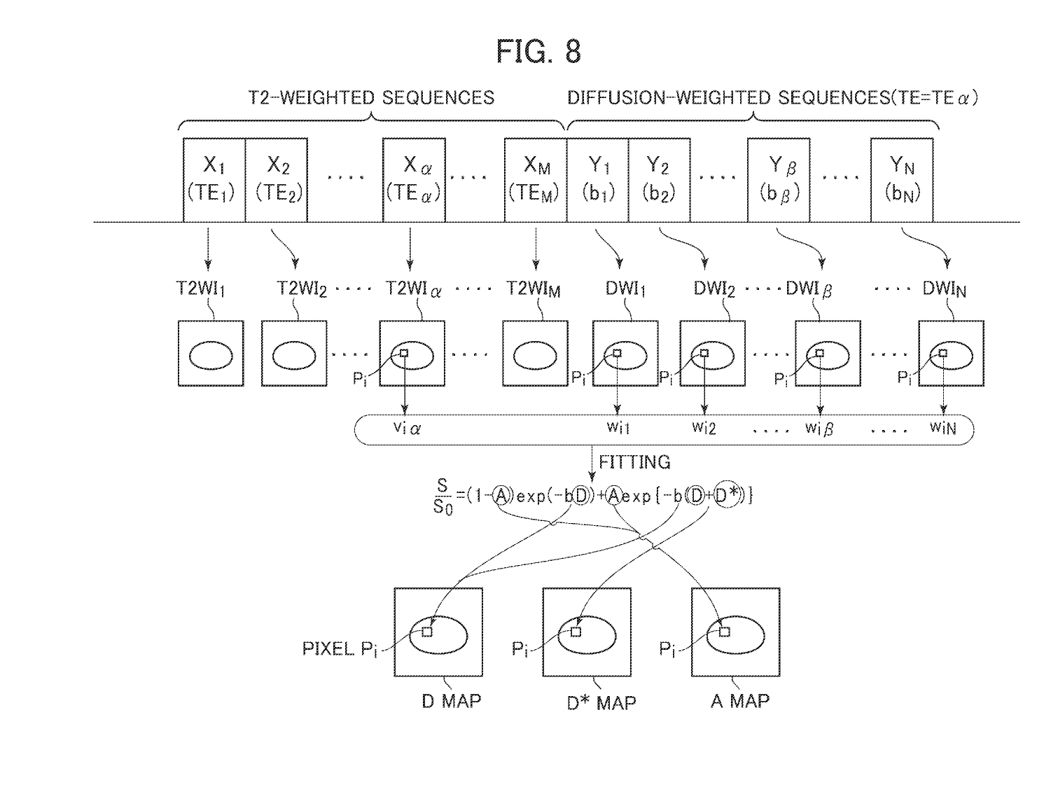

The M T2-weighted sequences X₁ to X_{M} are sequences for acquiring T2-weighted images T2WI₁ to T2WI_{M} of the slice SL. The M T2-weighted sequences X₁ to X_{M} are set to be different from one another in echo time TE. In FIG. 3, the echo times of the M T2-weighted sequences X₁ to X_{M} are respectively designated by symbols TE₁ to TE_{M}. The T2-weighted images T2WI₁ to T2WI_{M} obtained by the M T2-weighted sequences X₁ to X_{M} are used when a T2 map (see later described FIG. 6) is to be prepared. In addition, a T2-weighted image T2WI_{α} which is obtained by an α-th T2-weighted sequence X_{α} in the M T2-weighted sequences X₁ to X_{M} is used when D map, D* map and A map (see later described FIG. 8) are to be prepared. A method of preparing these maps will be described in detail later.

The diffusion-weighted sequences Y₁ to Y_{N} are sequences for acquiring diffusion-weighted images DWI₁ to DWI_{M} of the slice SL. The diffusion-weighted sequences Y₁ to Y_{N} have MPGs (Motion Probing Gradients) for obtaining diffusion-weighted images. In the exemplary embodiment, the b factor indicating the strength of the MPG is set to be b=b₁ to b_{N} for the respective diffusion-weighted sequences Y₁ to Y_{N}. The echo time TE for the diffusion-weighted sequences Y₁ to Y_{N} is set to an echo time TE_{α} which is the same as that of the T2-weighted X_{α}.

In the exemplary embodiment, images of the subject are acquired by using the T2-weighted sequences X₁ to X_{M} and the diffusion-weighted sequences Y₁ to Y_{N}. In the following, a flow when the images are to be acquired will be described.

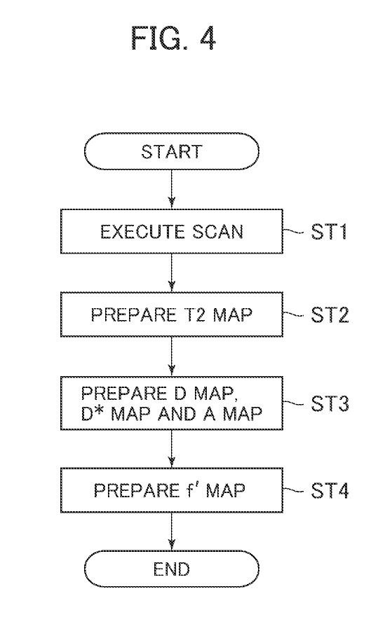

FIG. 4 is a diagram showing one example of the flow when the images are to be acquired in the exemplary embodiment.

In step ST1, the scan (the M T2-weighted sequences X₁ to X_{M} and the N diffusion-weighted sequences Y₁ to Y_{N}) shown in FIG. 3 is executed. The M T2-weighted images T2WI₁ to T2WI_{M} and the N diffusion-weighted images DWI₁ to DWI_{N} are obtained by the scan shown in FIG. 3. After the scan has been terminated, it proceeds to step ST2.

In step ST2, the T2 map is prepared. In the following, one example of a procedure of preparing the T2 map will be described.

In the exemplary embodiment, the T2 map is obtained by using a model that the signal value attenuates exponentially as TE is increased. This model is expressed by the following formula using T2.

S=S₀·exp(−TE·T2) Formula (6)

The following formula is obtained by logging the formula.

log(S)=log(S₀)−(TE·T2) Formula (7)

In the following, a method of preparing the T2 map will be described by using the model of the formula (7).

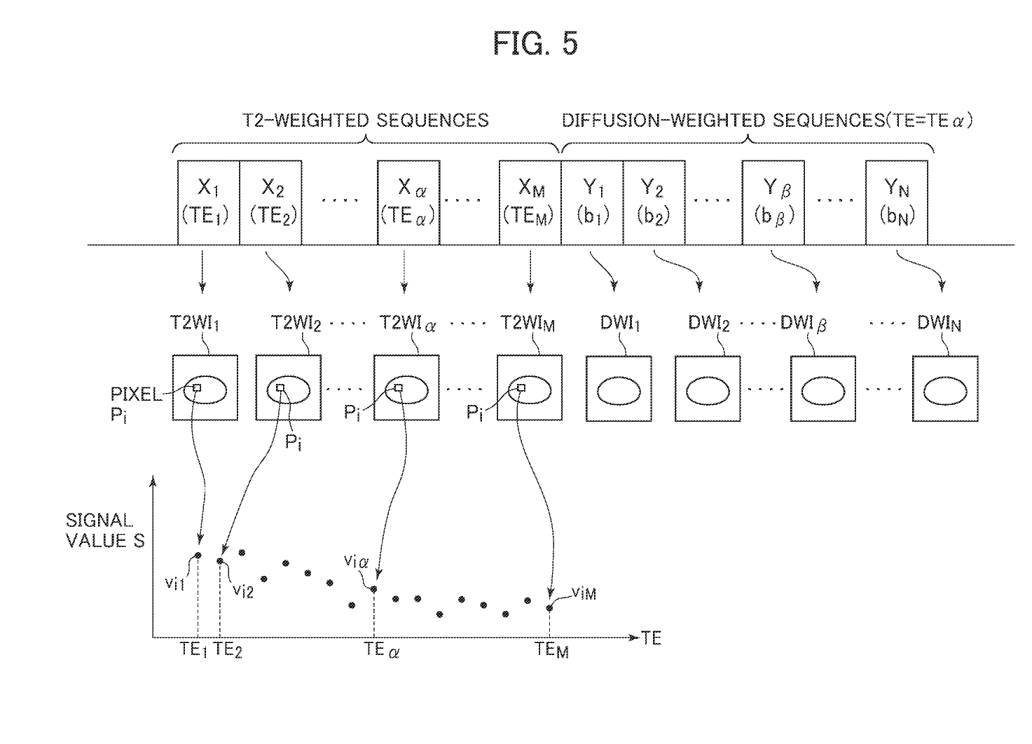

FIG. 5 and FIG. 6 are explanatory diagrams when the T2 map is to be prepared.

First, the first calculating unit 81 (see FIG. 1) distinguishes internal regions from external regions of the T2-weighted images T2WI₁ to T2WI_{M}. Since the pixel value of the pixel in the external region is sufficiently smaller than the pixel value of the pixel in the internal region, the external region can be distinguished from the internal region from a difference in pixel value.

Next, the first calculating unit 81 fetches signal values of pixels at the same positions from the internal regions of the T2-weighted images T2WI₁ to T2WI_{M}. The fetched signal values are schematically shown in FIG. 5. In FIG. 5, a pixel Pᵢ is regarded as the pixels at the same positions of the T2-weighted images T2WI₁ to T2WI_{M}. The first calculating unit 81 fetches signal values vᵢ₁ to v_{iM} of the pixels Pᵢ of the T2-weighted images T2WI₁ to T2WI_{M}.

The first calculating unit 81 performs fitting of the signal model expressed by the formula (7) on the basis of the fetched signal values vᵢ₁ to v_{iM} to calculate T2 in the formula (7) which fits the signal values vᵢ₁ to v_{iM} most. A state after the signal model expressed by the formula (7) has been subjected to fitting is schematically shown in FIG. 6. In FIG. 6, T2 which fits the signal values vᵢ₁ to v_{iM} most is indicated by T2=T2ᵢ.

T2=T2i so calculated is adopted as the T2 value of the pixel Pᵢ in the T2 map of the slice SL.

Although a method of obtaining the T2 value of the pixel Pi is shown in FIG. 5 and FIG. 6, T2 of another pixel included in the internal region can be also obtained by the same method. Therefore, the T2 map of the slice SL can be obtained. Although the M T2-weighted sequences are executed in order to prepare the T2 map in the exemplary embodiment, the value of M may be 2 or more. The T2 map can be prepared by executing two or more T2-weighted sequences which are different from one another in TE.

Incidentally, there are cases when each pixel in the T2-weighted images T2WI₁ to T2WI_{M} includes only the blood and there are also cases when it includes only the parenchyma other than the blood depending on the position of the pixel, and there are further cases when it includes components of both of the blood and the parenchyma. However, since most of the pixels are considerably large in percentage of the parenchyma in comparison with the blood, it can be thought that the T2 value of each pixel obtained by fitting is a value which is sufficiently close to the T2 value of the parenchyma. Therefore, it is estimated that the T2 value of each pixel of the T2 map is the T2 value of the parenchyma in the exemplary embodiment.

After the T2 map has been obtained, it proceeds to step ST3.

In step ST3, a map (a D map) of the diffusion coefficient D, a map (a D* map) of the diffusion coefficient D* and a map (an A map) of the coefficient A are prepared.

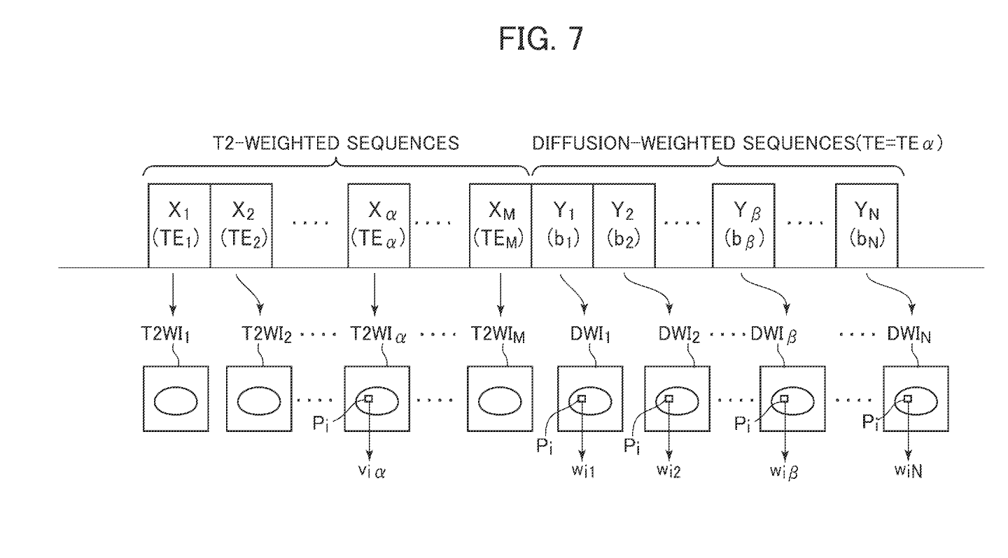

FIG. 7 and FIG. 8 are explanatory diagrams when the D map, the D* map and the A map are to be obtained.

The second calculating unit 82 (see FIG. 1) fetches signal values of pixels at the same positions from the internal region of the T2-weighted image T2WI_{α} and the internal regions of the N diffusion-weighted images DWI₁ to DWI_{N} of TE=TE_{α}. The fetched signal values are schematically shown in FIG. 7. In FIG. 7, the pixel Pᵢ is regarded as the pixels at the same positions. The second calculating unit 82 fetches the signal value V_{iα} of the pixel Pᵢ of the T2-weighted image T2WI_{α} and the signal values wᵢ₁ to w_{iN} of the pixels Pᵢ of the N diffusion-weighted images DWI₁ to DWI_{N}. Incidentally, since the echo time TE for the diffusion-weighted images DWI₁ to DWI_{N} is the echo time TE=TE_{α} which is the same as that of the T2-weighted image T2WI_{α}, these signal values v_{iα} and wᵢ₁ to w_{iN} are the signal values acquired at the same echo time TE. After these signal values v_{iα} and wᵢ₁ to w_{iN} have been fetched, D, D* and A are obtained by using the model of the formula (4).

The second calculating unit 82 performs fitting of the formula (4) using the signal values v_{iα} and wᵢ₁ to w_{iN} and calculates D, D* and A in the formula (4), which fit the signal values v_{iα} and wᵢ₁ to w_{iN} most. FIG. 8 schematically shows D, D* and A calculated by fitting. The D value of the pixel Pᵢ in the D map, the D* value of the pixel Pᵢ in the D* map and the A value of the pixel Pᵢ in the A map can be obtained by fitting.

Although a method of obtaining D, D* and A of the pixel Pᵢ is shown in FIG. 7 and FIG. 8, D, D* and A of another pixel included in the internal region can be also obtained by the same method. Therefore, the D map, the D* map and the A map of the slice SL can be obtained.

After these maps have been prepared, it proceeds to step ST4.

In step ST4, a map (an f′ map) of the perfusion fraction f′ is prepared on the basis of the formula (5).

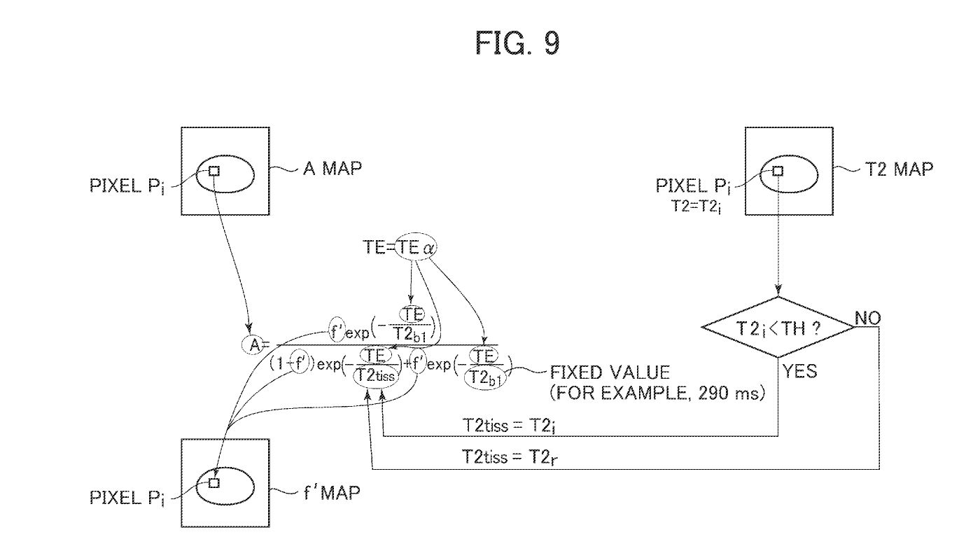

FIG. 9 is an explanatory diagram of a method of preparing the f′ map.

If the values of A, TE, T2_{bl}, T2ₜᵢₛₛ in the formula (5) are found, the perfusion fraction f′ can be obtained. A is calculated in step ST3, and TE is the TE value (TE_{α}) used in the diffusion-weighted sequences Y₁ to Y_{N}. Therefore, if the values of T2_{bl} and T2ₜᵢₛₛ are determined, the perfusion fraction f′ can be obtained. In the exemplary embodiment, the T2 value of the blood disclosed in a literature or the like is adopted as T2_{bl}. For example, T2_{bl}=290 ms. On the other hand, the value of T2ₜᵢₛₛ is determined on the basis of the T2 map obtained in step ST2. In the following, a method of determining the value of T2ₜᵢₛₛ will be described.

The third calculating unit 83 (see FIG. 1) determines whether the T2 value (T2) of the pixel Pᵢ in the T2 map is to be adopted as the value of T2ₜᵢₛₛ. This determination is performed by using a threshold value TH for determining whether the T2 value of the T2 map is to be adopted as T2ₜᵢₛₛ. The threshold value TH is set to a maximum value of the T2 values that the parenchyma in the slice SL could take. The third calculating unit 83 compares T2ᵢ of the pixel Pᵢ in the T2 map with the threshold value TH and determines whether it is to be adopted as the value of T2ₜᵢₛₛ on the basis of a result of comparison. Since the T2 values of the parenchymas included in the liver and its peripheral area are thought to be included in a range of about 10 ms to 100 ms, the value of the threshold value TH can be set to, for example, TH=150 ms. In a case where T2ᵢ<TH is established (yes), since T2ᵢ of the pixel Pᵢ in the T2 map is included in the range of the T2 values that the parenchyma could take, the third calculating unit 83 determines to adopt the T2ᵢ value of the pixel Pᵢ in the T2 map as the value of T2ₜᵢₛₛ On the other hand, in a case where T2ᵢ<TH is not established (no), since T2ᵢ of the pixel Pᵢ in the T2 map is out of the range of the T2 values that the parenchyma could take, the third calculating unit 83 determines not to adopt T2ᵢ of the pixel Pᵢ in the T2 map as the value of T2ₜᵢₛₛ. In a case where T2ᵢ is not adopted as the value of T2ₜᵢₛₛ, another value T2ᵣ which is smaller than the threshold value TH is adopted as the value of T2ₜᵢₛₛ in place of T2ᵢ. The value of T2ᵣ can be set to the mean value (for example, 60 ms) of the T2 values of the parenchyma included in the slice SL. In addition, in a case where the liver is regarded as important as an imaging object, the T2 value (about 50 ms) of the liver may be adopted as the value of T2ᵣ.

Therefore, since A, TE, T2_{bl}, T2ₜᵢₛₛ are determined, f′ can be obtained. Although a method of obtaining f′ of the pixel Pᵢ is shown in FIG. 9, f′ of another pixel included in the internal region can be also obtained by the same method.

After the f′ map has been prepared, the flow is terminated.

In the present embodiment, after the T2 map has been obtained by the T2-weighted sequences X₁ to X_{M} (step ST2), the A map (the map of the perfusion fraction that the T2 value is not taken into account) is obtained by the T2-weighted sequence X_{α} and the N diffusion-weighted images DWI₁ to DWI_{N} (step ST3). Then, the map (the f′map) of the perfusion fraction f′ that the T2 value has been taken into account is prepared on the basis of the T2 map and the A map (step ST4). Thus, the perfusion fraction which is small in error of estimation can be obtained.

In addition, since there is no need to obtain the T1 value in the present embodiment, there is no need to execute a scan for obtaining the T1 value. Therefore, an extension of the scan time can be minimized.

Incidentally, since in the present embodiment, a fixed value (for example, the literature value 290 ms) which has been set in advance is adopted as T2_{bl} of the blood, the perfusion fraction f′ is calculated with the same T2_{bl} value regardless of the subject. Thus, to what extent setting T2_{bl} to the fixed value affects the error of estimation of the perfusion fraction f′ becomes a problem. However, since it is thought that most of the pixels in each image are considerably large in percentage of the parenchyma in comparison with the blood, it is thought that the error of estimation of the perfusion fraction f′ can be sufficiently reduced even when the value of T2_{bl} is set to the fixed value.

Next, simulation was performed in order to compare the value of the perfusion fraction f′ to be calculated using the formula (5) with the value of the perfusion fraction f (see the formula (1)) to be calculated using the general IVIM model. In the following, results of the simulation will be described.

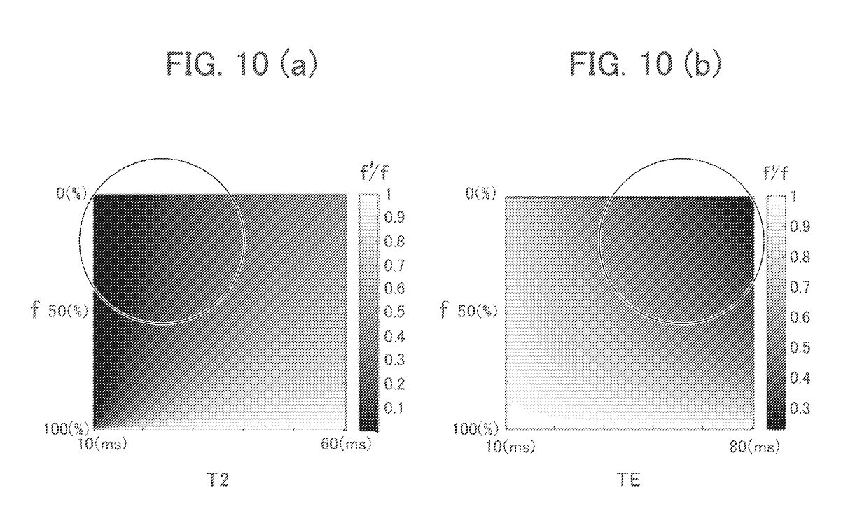

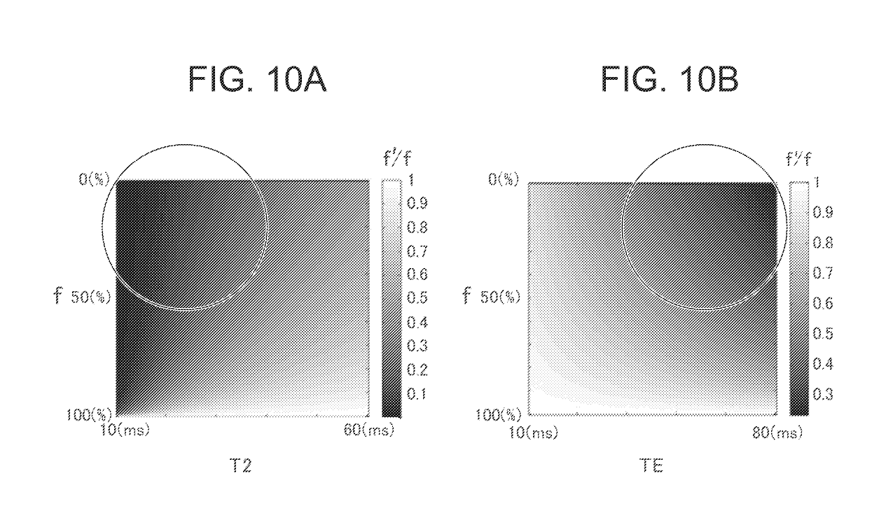

FIGS. 10A and 10B are diagrams showing the simulation results.

In FIGS. 10A and 10B, two graphs indicating a ratio f′/f of the two perfusion fractions are shown. FIG. 10A is one example of the graph that f′/f when TE=60 ms has been indicated with a gray scale, and the horizontal axis of the graph indicates the T2 value and the vertical axis indicates the perfusion fraction f (%). FIG. 10B is one example of the graph that f′/f when T2=46 ms has been indicated with a gray scale, and the horizontal axis of the graph indicates the TE value and the vertical axis indicates the perfusion fraction f (%).

It can be seen from FIG. 10A that in a case where the value of f is small and the T2 value is small, the ratio f′/f is lowered. In addition, it can be seen from FIG. 10B that in a case where the value of f is small and the TE value is large, the ratio f′/f is lowered. Therefore, it can be seen that the advantage that the perfusion fraction has been calculated by taking T2 into account is greatly exhibited in encircled regions in the graphs.

Many widely different embodiments may be configured without departing from the spirit and the scope of the present invention. It should be understood that the present invention is not limited to the specific exemplary embodiments described in the specification, except as defined in the appended claim.

1. A magnetic resonance apparatus comprising:
a scanning unit configured to execute a plurality of T2-weighted sequences having different echo times, and a plurality of diffusion-weighted sequences having different b factors;
a first calculating unit configured to calculate a T2 value based on a plurality of T2-weighted images obtained by the plurality of T2-weighted sequences;
a second calculating unit configured to calculate a perfusion fraction where the T2 value is not taken into account based on a first T2-weighted image in the plurality of T2-weighted images and a plurality of diffusion-weighted images obtained by the plurality of diffusion-weighted sequences; and
a third calculating unit configured to calculate a perfusion fraction where the T2 value has been taken into account based on the T2 value calculated by the first calculating unit, and based on the perfusion fraction calculated by the second calculating unit.

2. The magnetic resonance apparatus of claim 1, wherein the third calculating unit is configured to calculate the perfusion fraction where the T2 value has been taken into account by using a relational expression that defines a relation among the perfusion fraction where the T2 value is not taken into account, the T2 value, and the perfusion fraction where the T2 value has been taken into account.

3. The magnetic resonance apparatus of claim 2, wherein the relational expression includes a T2 value of a parenchyma and a T2 value of blood as the T2 value, and wherein the third calculating unit is configured to use the T2 value that the first calculating unit has calculated as the T2 value of the parenchyma, and configured to use a T2 value other than the T2 value that the first calculating unit has calculated as the T2 value of the blood.

4. The magnetic resonance apparatus of claim 3, wherein the third calculating unit is configured to compare the T2 value that the first calculating unit has calculated with a threshold value, and configured to decide whether the T2 value that the first calculating unit has calculated is to be used as the T2 value of the parenchyma based on a result of the comparison.

5. The magnetic resonance apparatus of claim 1, wherein the perfusion fraction where the T2 value is not taken into account and the perfusion fraction where the T2 value has been taken into account are calculated by using a model expressed by the following formula:
$$\frac{S}{{S}_{0}} = ( 1 - A ) exp ( - bD ) + A exp ( - b ( D + {D}^{*} ) ) \\ [ [ Here ] ] \underline{where}$$
$$A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )}$$
and where S is a Signal value with an arbitrary b factor, S₀ is a Signal value when b=0, D is an Apparent diffusion coefficient by a true diffusion component, D* is an Apparent diffusion coefficient by a pseudo diffusion component, A is a Perfusion fraction where the T2 value is not taken into account, f′ is a Perfusion fraction where the T2 value has been taken into account, TE is an Echo time, T2ₜᵢₛₛ is a T2 value of a parenchyma, and T2_{bl} is a T2 value of blood.

6. The magnetic resonance apparatus of claim 2, wherein the perfusion fraction where the T2 value is not taken into account and the perfusion fraction where the T2 value has been taken into account are calculated by using a model expressed by the following formula:
$$\frac{S}{{S}_{0}} = ( 1 - A ) exp ( - bD ) + A exp ( - b ( D + {D}^{*} ) ) \\ [ [ Here ] ] \underline{where}$$
$$A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )}$$
and where S is a Signal value with an arbitrary b factor, S₀ is a Signal value when b=0, D is an Apparent diffusion coefficient by a true diffusion component, D* is an Apparent diffusion coefficient by a pseudo diffusion component, A is a Perfusion fraction where the T2 value is not taken into account, f′ is a Perfusion fraction where the T2 value has been taken into account, TE is an Echo time, T2ₜᵢₛₛ is a T2 value of a parenchyma, and T2_{bl} is a T2 value of blood.

7. The magnetic resonance apparatus of claim 3, wherein the perfusion fraction where the T2 value is not taken into account and the perfusion fraction where the T2 value has been taken into account are calculated by using a model expressed by the following formula:
$$\frac{S}{{S}_{0}} = ( 1 - A ) exp ( - bD ) + A exp ( - b ( D + {D}^{*} ) ) \\ [ [ Here ] ] \underline{where}$$
$$A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )}$$
and where S is a Signal value with an arbitrary b factor, S₀ is a Signal value when b=0, D is an Apparent diffusion coefficient by a true diffusion component, D* is an Apparent diffusion coefficient by a pseudo diffusion component, A is a Perfusion fraction where the T2 value is not taken into account, f′ is a Perfusion fraction where the T2 value has been taken into account, TE is an Echo time T2ₜᵢₛₛ is a T2 value of a parenchyma, and T2_{bl} is a T2 value of blood.

8. The magnetic resonance apparatus of claim 4, wherein the perfusion fraction where the T2 value is not taken into account and the perfusion fraction where the T2 value has been taken into account are calculated by using a model expressed by the following formula:
$$\frac{S}{{S}_{0}} = ( 1 - A ) exp ( - bD ) + A exp ( - b ( D + {D}^{*} ) ) \\ [ [ Here ] ] \underline{where}$$
$$A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )}$$
and where S is a Signal value with an arbitrary b factor, S₀ is a Signal value when b=0, D is an Apparent diffusion coefficient by a true diffusion component, D* is an Apparent diffusion coefficient by a pseudo diffusion component, A is a Perfusion fraction where the T2 value is not taken into account, f′ is a Perfusion fraction where the T2 value has been taken into account, TE is an Echo time T2ₜᵢₛₛ is a T2 value of a parenchyma, and T2_{bl} is a T2 value of blood.

9. A method for calculating a perfusion fraction, the method comprising:
calculating a T2 value based on a plurality of T2-weighted images obtained by a plurality of T2-weighted sequences having different echo times;
calculating a perfusion fraction where the T2 value is not taken into account based on a first T2-weighted image in the plurality of T2-weighted images, and a plurality of diffusion-weighted images obtained by a plurality of diffusion-weighted sequences having different b factors; and
calculating a perfusion fraction where the T2 value has been taken into account based on the T2 value calculated by the first calculation step, and based on the perfusion fraction calculated by the second calculation step.

10. The method of claim 9, wherein calculating a perfusion fraction where the T2 value has been taken into account comprises calculating the perfusion fraction where the T2 value has been taken into account by using a relational expression that defines a relation among the perfusion fraction where the T2 value is not taken into account, the T2 value, and the perfusion fraction where the T2 value has been taken into account.

11. The method of claim 10, wherein the relational expression includes a T2 value of a parenchyma and a T2 value of blood as the T2 value.

12. The method of claim 9, further comprising:
comparing the T2 value with a threshold value; and
deciding whether the T2 value is to be used as a T2 value of a parenchyma based on a result of the comparison.

13. The method of claim 9, wherein the perfusion fraction where the T2 value is not taken into account and the perfusion fraction where the T2 value has been taken into account are calculated by using a model expressed by the following formula:
$$\frac{S}{{S}_{0}} = ( 1 - A ) exp ( - bD ) + A exp ( - b ( D + {D}^{*} ) )$$
$$where$$
$$A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )}$$
and where S is a Signal value with an arbitrary b factor, S₀ is a Signal value when b=0, D is an Apparent diffusion coefficient by a true diffusion component, D* is an Apparent diffusion coefficient by a pseudo diffusion component, A is a Perfusion fraction where the T2 value is not taken into account, f′ is a Perfusion fraction where the T2 value has been taken into account, TE is an Echo time, T2ₜᵢₛₛ is a T2 value of a parenchyma, and T2_{bl} is a T2 value of blood.

14. A computer for use with a magnetic resonance apparatus, said computer comprising:
a first calculating unit configured to calculate a T2 value based on a plurality of T2-weighted images obtained by a plurality of T2-weighted sequences having different echo times;
a second calculating unit configured to calculate a perfusion fraction where the T2 value is not taken into account based on a first T2-weighted image in the plurality of T2-weighted images and a plurality of diffusion-weighted images obtained by a plurality of diffusion-weighted sequences having different b factors; and
a third calculating unit configured to calculate a perfusion fraction where the T2 value has been taken into account based on the T2 value calculated by the first calculating unit, and based on the perfusion fraction calculated by the second calculating unit.

15. The computer of claim 14, wherein the third calculating unit is configured to calculate the perfusion fraction where the T2 value has been taken into account by using a relational expression that defines a relation among the perfusion fraction where the T2 value is not taken into account, the T2 value, and the perfusion fraction where the T2 value has been taken into account.

16. The computer of claim 15, wherein the relational expression includes a T2 value of a parenchyma and a T2 value of blood as the T2 value, and wherein the third calculating unit is configured to use the T2 value that the first calculating unit has calculated as the T2 value of the parenchyma, and configured to use a T2 value other than the T2 value that the first calculating unit has calculated as the T2 value of the blood.

17. The computer of claim 16, wherein the third calculating unit is configured to compare the T2 value that the first calculating unit has calculated with a threshold value, and configured to decide whether the T2 value that the first calculating unit has calculated is to be used as the T2 value of the parenchyma based on a result of the comparison.

18. The computer of claim 14, wherein the perfusion fraction where the T2 value is not taken into account and the perfusion fraction where the T2 value has been taken into account are calculated by using a model expressed by the following formula:
$$\frac{S}{{S}_{0}} = ( 1 - A ) exp ( - bD ) + A exp ( - b ( D + {D}^{*} ) )$$
$$where$$
$$A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )}$$
and where S is a Signal value with an arbitrary b factor, S₀ is a Signal value when b=0, D is an Apparent diffusion coefficient by a true diffusion component, D* is an Apparent diffusion coefficient by a pseudo diffusion component, A is a Perfusion fraction where the T2 value is not taken into account, f′ is a Perfusion fraction where the T2 value has been taken into account, TE is an Echo time, T2ₜᵢₛₛ a T2 value of a parenchyma, and T2_{bl} is a T2 value of blood.

19. The computer of claim 15, wherein the perfusion fraction where the T2 value is not taken into account and the perfusion fraction where the T2 value has been taken into account are calculated by using a model expressed by the following formula:
$$\frac{S}{{S}_{0}} = ( 1 - A ) exp ( - bD ) + A exp ( - b ( D + {D}^{*} ) )$$
$$where$$
$$A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )}$$
and where S is a Signal value with an arbitrary b factor, S₀ is a Signal value when b=0, D is an Apparent diffusion coefficient by a true diffusion component, D* is an Apparent diffusion coefficient by a pseudo diffusion component, A is a Perfusion fraction where the T2 value is not taken into account, f′ is a Perfusion fraction where the T2 value has been taken into account, TE is an Echo time, T2ₜᵢₛₛ is a T2 value of a parenchyma, and T2_{bl} is a T2 value of blood.

20. The computer of claim 16, wherein the perfusion fraction where the T2 value is not taken into account and the perfusion fraction where the T2 value has been taken into account are calculated by using a model expressed by the following formula:
$$\frac{S}{{S}_{0}} = ( 1 - A ) exp ( - bD ) + A exp ( - b ( D + {D}^{*} ) )$$
$$where$$
$$A = \frac{{f}^{′} exp ( - \frac{TE}{T {2}_{b 1}} )}{( 1 - {f}^{′} ) exp ( - \frac{TE}{T {2}_{tiss}} ) + {f}^{′} exp ( - \frac{TE}{T {2}_{b 1}} )}$$
and where S is a Signal value with an arbitrary b factor, S₀ is a Signal value when b=0, D is an Apparent diffusion coefficient by a true diffusion component, D* is an Apparent diffusion coefficient by a pseudo diffusion component, A is a Perfusion fraction where the T2 value is not taken into account, f′ is a Perfusion fraction where the T2 value has been taken into account, TE is an Echo time, T2ₜᵢₛₛ is a T2 value of a parenchyma, and T2_{bl} is a T2 value of blood.
